(19) United States
(12) Patent Application Publication
(10) Pub. No.: US 2017/0049336 A1
(43) Pub. Date: Feb. 23, 2017

(54) PHYSIOLOGICAL SENSORS, SYSTEMS, KITS AND METHODS THEREFOR

(71) Applicant: REVEAL BIOSENSORS, INC., San Jose, CA (US)

(72) Inventor: Guy Meredith HATCH, Logan, UT (US)

(21) Appl. No.: 15/307,152

(22) PCT Filed: Apr. 29, 2015
(86) PCT No.: PCT/US2015/028198

§ 371 Date: Oct. 27, 2016

Related U.S. Application Data
(60) Provisional application No. 61/987,027, filed on May 1, 2014.
(60) Provisional application No. 61/987,015, filed on May 1, 2014.

(51) Int. Cl. A61B 5/0205; A61B 5/1455; G06F 19/00; A61B 5/00; H04W 4/14; H04L 12/58; A61B 5/0456; A61B 5/024

(52) U.S. Cl. CPC A61B 5/0205; A61B 5/0456; A61B 5/14552; A61B 5/02427; A61B 5/02433; A61B 5/4869; A61B 5/6833; A61B 5/6823; A61B 5/6824; A61B 5/0022; H04W 4/14; H04L 51/08; G06F 19/3406

(57) ABSTRACT

A combination physiologic status sensor system is described. It integrates a highly efficient reflectance oximeter, acquisition of a 2-lead electrocardiogram (“ECG”) heart rate signal, a photonic indicator of molecular products of anaerobic metabolism, and a skin temperature sensor. Placement of this motion tolerant sensor system may be on the adult or child subject's upper arm or infant chest, or chest and abdomen, for acquisition of high quality signals. Efficient use of battery power, combined optionally with wireless communications, enables continuous ambulatory use for extended periods of time. The comprehensive physiologic profile provided by this multi-factorial sensor and analysis system is anticipated to be of benefit in providing physiologically important, actionable information in a wide range of applications, including athletic training and performance, high risk occupational safety monitoring, and critical and convalescent medical care of patients of all ages and sizes.

### CROSS-REFERENCE

This application claims the benefit of U.S. Provisional Application Nos. 61/987,027, filed May 1, 2014, entitled REFLECTANCE OXIMETER ELECTRONIC INTERFACE, and 61/987,015 filed May 1, 2014, entitled REFLECTANCE OXIMETER OPTICS which applications are incorporated herein by reference.

### BACKGROUND

Currently available medical monitoring technology measures a number of individual parameters of physiology as separate information entities; leaving clinicians to mentally integrate the contextual significance of the measured parameters. For example, one of the primary life-dependencies of humans is continuous delivery of oxygen by the lungs and circulatory system to all tissues of the body in sufficient quantity to maintain efficient aerobic metabolism. Breathing rate, heart rate, cardiac output, laboratory measurement of arterial blood hemoglobin level and “blood gas,” or oxygen saturation (SaO₂) level, and arterial blood flow distribution are all significant factors in this process; each potentially being measured or monitored separately with currently available technology.

One such suitable non-invasive monitoring device is a photoplethysmogram, which is an instrument for measuring changes in volume within an organ resulting from fluctuations in the amount of blood or air it contains. A photoplethysmogram (“PPG”) is an optically obtained plethysmogram. A PPG is often obtained by using a pulse oximeter that illuminates the skin and measures changes in light absorption. A conventional pulse oximeter monitors the perfusion of blood to the dermis and subcutaneous tissue of the skin.

Fingertip pulse oximetry (“pulse ox”) was initially introduced as a continuous, non-invasive alternative to periodic arterial blood sampling for laboratory measurement of blood gas parameters. The fingertip pulse oximeter sensor has now become ubiquitous in modern medical care. However, when a patient is fully “wired,” e.g., with electrocardiograph (“ECG”) leads and a pulse oximeter sensor, the patient's mobility is hampered. Moreover, even slight movement of the fingertip pulse oximeter sensor may result in false alarms, or in delayed or missed real alarms, depending on which signal processing method is employed. Alternative attempts at reflectance oximetry, for example, with a sensor placed on the subject's forehead or chest, have been only marginally successful for a variety of reasons. The size of the battery needed to supply the relatively high power requirement of pulse oximeter light emitting diodes (“LEDs”) has further limited the usefulness of pulse oximetry for ambulatory and home care monitoring.

Adding to the complexity of the innovation challenge are the unique needs and small body size of prematurely born newborn infants with critical health problems. Available pulse oximetry devices most commonly pass red and infrared light through body tissue, such as a fingertip, or an infant's hand or foot, and measure the variation in absorbance of the light relative to the vascular blood volume pulsation created by the heart pumping cycle. However, the optical and human interface components of currently available sensors, as typically applied to the subject's fingertip, have the following limitations:
Limited to less physiologically indicative sensor application sites;
Sensor vs. skin motion-induced distortion of the optical signal;
Signal distortion and interference from environmental electromagnetic noise;
Delayed alarms, false alarms, and missed real alarms;
Inefficient use of electrical power;
Poorly compatible with “wearable” monitoring formats; and
Poorly applicable to the unique needs and body interface of premature infants.

The limited range of tissue penetration of the typical wavelengths used with currently available pulse oximeter sensors requires sensors to be applied across sites no more than about ½ inch thick; thus the selection of the fingertip. However, reduced peripheral perfusion, in reflex response to severe physiologic stress, reduces or eliminates the pulsating optical signal at the ends of the arms and legs. This response inherently compromises sensor function during the very pathologies for which physiologic monitoring is most needed; i.e. shock, and lung diseases, such as chronic obstructive pulmonary disease (“COPD”), pneumonia, asthma and premature newborn respiratory distress syndrome. Alternative designs, such as those applied to the subject's forehead or nasal bridge, are limited by internal shunting of light between emitters and sensor.

The mechanical instability of the fingertip location creates sensor vs. skin motion-generated signal artifacts during normal activity. Since the computed peripheral arteriolar oxygen saturation (“SpO₂”) is typically based on the very small (0.5-1.0%) alternating current (“AC”) portion of the optical signal, even slight signal distortion can become problematic. Normal breathing may also cause up to 30% direct current (“DC”) deviation of the fingertip oximeter signal. Since computing the SpO₂ value is dependent upon continuous availability of a strong signal, noise-inducing sensor movements regularly interrupt the process and can trigger a false alarm. Clinicians using such equipment tend to become less responsive as they experience many false alarms. Masimo SET® pulse oximeters (available from Masimo Corporation, Irvine, Calif.) use Fast Fourier Transform (“FFT”) signal processing in an attempt to separate the desired features of the regularly occurring cardiac-cycle-induced optical pulse waveform from the more random noise in the raw signal. This method, however, removes potentially useful features from the signal and increases alarm response delay.

The relatively high current drawn by available pulse oximeter LEDs, combined with the length of the sensor cable, results in large switching transients that require extended illumination periods to allow the light level to stabilize for each data sample. The high electrical impedance of photodiode detectors also makes connecting cables vulnerable to electromagnetic interference (“EMI”).

Oximetry measurement at the end of an extremity (e.g., at an adult's fingertip or an infant's foot) maximizes the time delay between a central body change in arterial blood oxygen content; and sensor detection of that change. As a result, alarm annunciation for the purpose of summoning assistance to a patient with a physiologic crisis is also delayed.

The continuous cycling of the LEDs in currently available pulse oximetry devices and related pulse rate monitoring devices requires more electrical power than can be conveniently and comfortably supplied with batteries for useful durations of ambulatory monitoring of chronically ill patients that could benefit from such monitoring, for example, patients suffering from COPD and heart failure.

Additionally, fingertip pulse oximetry cannot be effectively performed outside the clinical setting, e.g., for athletes, astronauts, divers, military personnel, and others involved in vigorous, potentially hazardous work or recreational activities using their hands.

Additionally, many unique monitoring challenges are experienced by premature infants and those caring for them. These challenges have, to date, been either ignored or only marginally addressed with minor modifications of pulse oximeter sensors initially designed and developed for use on adult fingertips. For example, since the baby's hands and feet are the only places where such “adult-style” sensors can be mounted, the inevitable and random ‘wiggles’ of the baby are a major challenge to signal stabilization. These physiological sites are also compromised by peripheral vasoconstriction during physiologic or disease crisis. Further, current monitoring technology does not monitor the onset of, and/or continuously track the severity of, potentially life-threatening hemodynamic shunting, which is largely unique to premature infants vs. older children and adults. All newborn infants must successfully adapt from fetal circulation through a placenta to air breathing to meet the metabolic needs of their vital organs for oxygen. However, the uncertainty of the varying “correct” blood oxygen saturation through this transition period lacks a reliable and continuous index of the sufficiency of oxygen delivery to vital tissues. If too little oxygen is delivered, the brain, heart, gut, kidneys and other vital organs can be irreparably damaged, possibly even causing death. Too much oxygen delivery may result in severe damage to the infant's retinas, resulting in vision impairment or even total blindness.

What is needed is a device that is convenient to use that can be used for measuring one or more biological parameters indicating, among other useful information, the sufficiency of oxygen delivery to vital tissues in infants and adults without impeding user mobility and without the quality of the signal being compromised by movement.

### SUMMARY

Disclosed is a physiologic sensor that is suitable for placement on the upper arm of an adult or child, or on the chest or abdomen of an infant. The device is positionable with straps or adhesive patches, or integratable into clothing in a “wearable” format. The sensor is designed for placement on the chest or upper arm of a subject where sensor motion artifact is less likely to occur with normal activity than on an adult or child fingertip or infant hand or foot.

The sensor is configurable to detect increasing levels of molecular products of anaerobic metabolism in the skin by assessing a combination of a decreasing DC photocurrent response from a first wavelength illumination and a rising DC photocurrent response from a second wavelength illumination, both of which are correlated with progressively deeper hypoxia of the illuminated skin tissue.

The device can integrate an ECG skin surface electrode pair and provide signal pre-amplification within the sensor. ECG skin potential is sensed between the sensor cover plate, which is electrically connected to sensor ground, and an adjacently-located second skin contact electrode, whose differential cardiac bio-potential AC signal is input to the pre-amplifier via one of the sensor solder pads. The leading edge of the derived ECG R-wave is used to generate a timing trigger pulse that is used to control oximeter sample timing and to provide ECG heart rate data output.

The device also provides an internal logic voltage-to-LED-current conversion circuit to perform LED power switching within the sensor housing to minimize the powered interval needed for data sampling. A trans-impedance amplification circuit can be provided adjacent to the photodiode detector to condition the high impedance photocurrent signal and to reduce susceptibility to EMI.

Electrical power efficiency of the present device sensor system is enhanced by use of selectively timed sampling of the PPG. This is achieved by briefly powering the oximeter LEDs at only the “trough” and “peak” of each cardiac cycle instead of continuously sampling throughout the cardiac cycle as is done with currently available pulse oximetry systems. Pulse oximeters continuously cycle their red and infrared LEDs, with brief ‘off’ periods in between to detect ambient light interference. This results in the LEDs being powered on about half of the time and consuming up to 40 milliamps while turned on. This configuration typically requires the average continuous power consumption of up to about 20 milliamps. LEDs consume the majority of the electrical power needed to operate pulse oximeter sensors and this power must be supplied by a battery if the monitor system is not attached to an AC power source. However, the data produced by this cycling process during pulse oximetry is derived by detecting and capturing only the “maximum” and “minimum” values of the continuous data stream. These two very brief time period segments of the continuous waveform signal, known as the “trough” and the “peak,” comprise a total of less than 5% of the cardiac cycle waveform. The remaining 95% of the LED power consumed by pulse oximeters is wasted, since it does not contribute to the generation of either the data output or the alarm function. Many clinicians like to periodically see the full waveform on the monitor display so they can subjectively evaluate the quality of the signal. However, signal quality can be more objectively measured by analysis of the “trough” and “peak” data. In situations where power consumption is judged to be less of an issue, the full waveform can be obtained and displayed, or periodically obtained and recorded for later review. The disclosed devices are configured to define a minimum LED power requirement for production of fully valid oximetry data and alarm response in order to maximize the power efficiency of the system for battery-supported use.

Light shunting directly from emitters to a sensor within the housing is prevented by any suitable visible and IR opaque material barrier, such as metal, between the LED light emitters and the light detector. An aperture filled with optically clear epoxy over the emitters, projects light into the subject's skin. Lateral to the emitter aperture is a second aperture, which can also be filled with optically clear epoxy, which admits tissue-interacted diffused light to a photodiode for monitoring subcutaneous arteriolar hemoglobin/oxygen saturation, or SpO₂ and for detecting changes in the reflected DC photocurrent resulting from emission of two wavelengths.

A center-to-center lateral separation between the LED light emitter aperture and the photodiode sensor aperture of the device can be between 5 mm and 9 mm. The device is configured to generate two wavelengths that are used in time-alternating fashion in oximetry to relate to the different levels of spectral absorbance of reduced hemoglobin, vs. oxygenated hemoglobin, which is maximized at about 660 nm, and which appears visually as red. In an exemplar embodiment, two LEDs are provided. One LED emitting a first wavelength and a second LED emitting a second wavelength mounted close together beneath an emitter aperture. Alternating with the red LED, a second LED, emitting in the non-visible infrared portion of the spectrum, is typically used as a general light absorbance reference to help compensate for variations in skin pigment and tissue absorbance and to diminish the effect of variations in the presence of non-pulsatile capillary and venous blood. Fingertip pulse oximetry typically uses about 940 nm center wavelength infrared LEDs, likely because this wavelength region is near the maximum sensitivity wavelength of the silicon photodiode sensors used. The sensor photodiode is selected to respond adequately to both wavelengths used and produces an output photocurrent level in response to each LED operating at alternating times. Background illumination is typically sensed between the activation of the individual LEDs and the proximal time photocurrent level subtracted from either the preceding or the following LED-illuminated photocurrent value to minimize the effect of ambient light interference.

Skin temperature of a user can be sensed with, for example, a thermistor mounted within the sensor housing and connected electrically between the sensor circuit ground and one of the solder pads on the bottom of the sensor circuit. This arrangement allows periodic, very brief signal sampling, while minimizing self-heating of the thermistor.

The device provides feedback that can be used to guide manual regulation, or to automate regulation of oxygen concentration in breathing gas during care of premature infants, during surgical anesthesia and during weaning from mechanically assisted ventilation and/or oxygen supplementation of the breathing gas.

The device is configurable to be compatible with minimal cost methods of modern automated mass-production of electrical and photonic components and systems.

An aspect of the disclosure is directed to devices comprising: a first means for emitting a first wavelength wherein the first means for emitting a first wavelength is configurable to emit a first target wavelength during a trough or a peak determined by an ECG R-wave triggered timing interval; a second means for emitting a second wavelength wherein the second means for emitting a second wavelength is configurable to emit a second target wavelength during the trough or the peak determined by the ECG R-wave triggered timing interval; a detection means optically isolated from the first means for emitting the first wavelength and the second means for emitting the second wavelength; and a processor means configured to receive an input from the detection means. Devices additionally are powered by a suitable power supply means. Additionally, devices are configurable so that the first target wavelength is a red wavelength and the second target wavelength is an infrared wavelength. Suitable configurations include a configuration where the first target center wavelength is 660 nm and the second target center wavelength is 805 nm. Additionally, devices can further comprises a data transmitter means. In some configurations, the devices are configurable to detect one or more of reflectance oximetry, and anaerobic metabolism. Additionally, a suitable housing means can be provided, for example a housing means having a first aperture and a second aperture. The first aperture and the second aperture can be filled with an optically clear material. Additionally, devices can include an ECG R-wave pre-amplifier circuit means. A securer means configured to secure the device to an arm or a chest of a user can also be provided. One or more electrically conductive skin contact adhesive means can also be provided.

Another aspect of the disclosure is directed to devices comprising: a first LED emitter wherein the first LED emitter is configurable to emit a first target wavelength during a trough or a peak determined by an ECG R-wave triggered timing interval; a second LED emitter wherein the second LED emitter is configurable to emit a second target wavelength during the trough or the peak determined by the ECG R-wave triggered timing interval; a detector optically isolated from the first LED emitter and the second LED emitter; and a processor configured to receive an input from the detector. Suitable power may also be provided to the devices. In some configurations, the first target center wavelength is a red wavelength and the second target center wavelength is an infrared wavelength. The first target center wavelength can be 660 nm and the second target center wavelength can be 805 nm. Devices can further comprise a data transmitter. Additionally, the device is configurable to detect one or more of reflectance oximetry, and anaerobic metabolism. A housing having a first aperture and a second aperture can also be provided. Additionally, the first aperture and the second aperture are filled with an optically clear material. Some configurations may also include one or more of an ECG R-wave pre-amplifier circuit, a securer configured to secure the device to an arm or a chest of a user, and/or one or more electrically conductive skin contact adhesive pads.

Yet another aspect of the disclosure is directed to devices comprising: a housing adapted to engage a chest or an arm of a user wherein the housing has a first aperture and a second aperture; a first LED emitter wherein the first LED emitter is configurable to emit a first target center wavelength during a trough or a peak determined by an ECG R-wave triggered timing interval through the first aperture; a second LED emitter wherein the second LED emitter is configurable to emit a second target center wavelength during the trough or the peak determined by the ECG R-wave triggered timing interval through the first aperture; a detector disposed on an adjacent plate to the LED emitter within the housing wherein the detector is optically isolated in the housing from the first LED emitter and the second LED emitter and adjacent the second aperture; and a processor configured to receive an input from the detector. The first target wavelength can be a red wavelength and the second target wavelength can be an infrared wavelength. In some configurations, the first target center wavelength is 660 nm and the second target center wavelength is 805 nm. At least some configurations can also include a data transmitter. Additionally, the device is configurable to detect one or more of reflectance oximetry, and anaerobic metabolism. The first aperture and the second aperture can be filled with an optically clear material. Additionally, in some configurations, an ECG R-wave pre-amplifier circuit can be provided. A securer can be provided which is configured to secure the device to the arm or the chest of the user.

Still another aspect of the disclosure is directed to a method of detecting a biological parameter comprising: placing a device in contact with an arm or a chest of a patient wherein the device further comprises, a first LED emitter wherein the first LED emitter is configurable to emit a first target center wavelength during a trough or a peak determined by an ECG R-wave timing trigger and derived timing interval, a second LED emitter wherein the second LED emitter is configurable to emit a second target center wavelength during the trough or the peak determined by the ECG R-wave timing trigger and derived timing interval, a detector optically isolated from the first LED emitter and the second LED emitter, a processor configured to receive an input from the detector, powering the device with a power supply; emitting a light in a first wavelength and alternately emitting a light in a second wavelength, wherein the emitted lights are selectively emitted during the trough or peak determined by the ECG-R-wave; detecting a reflected light; and analyzing the detected signal produced by the reflected light. Additionally, the method can include the step of determining a reflectance oximetry value for the patient. Still other aspects of the method can include the step of determining an index of anaerobic metabolism of the sensor-illuminated skin of the patient. Moreover, data from the device can be transmitted to a second device.

Another aspect of the disclosure is directed to a communication system, comprising: a detection device having a first LED emitter wherein the first LED emitter is configurable to emit a first target wavelength during a trough or a peak determined by an ECG R-wave, a second LED emitter wherein the second LED emitter is configurable to emit a second target wavelength during the trough or the peak determined by the ECG R-wave, a detector optically isolated from the first LED emitter and the second LED emitter, and a detection device processor configured to receive an input from the detector; a power supply in communication with the detection device to power the detection device; a server computer system; a measurement module on the server computer system for permitting a transmission of a measurement from the detection device over a network; and at least one of an API engine connected to at least one of the detection device to create a message about the measurement and transmit the message over an API integrated network to a recipient having a predetermined recipient user name, an SMS engine connected to at least one of a system for detecting physiological parameters and the detection device to create an SMS message about the measurement and transmit the SMS message over the network to a recipient device having a predetermined measurement recipient telephone number, or an email engine connected to at least one of the detection device to create an email message about the measurement and transmit the email message over the network to a recipient email having a predetermined recipient email address. The system can also have a storing module on the server computer system for storing the measurement in a detection device server database. The detection device can be connectable to the server computer system over at least one of a mobile phone network or an Internet network, and a browser on a measurement recipient electronic device is used to retrieve an interface on the server computer system. In at least some configurations, an interface on the server computer system, the interface being retrievable by an application on a mobile device. Additionally, the server computer system can be connectable over a cellular phone network to receive a response from a measurement recipient mobile device. Some system configurations also include a downloadable application residing on a measurement recipient mobile device, the downloadable application transmitting a response and a measurement recipient phone number ID over a cellular phone network to the server computer system, the server computer system utilizing the measurement recipient phone number ID to associate the response with an SMS measurement and/or a transmissions module that transmits the measurement over a network other than a cellular phone SMS network to a measurement recipient user computer system, in parallel with the measurement that is sent over the cellular phone SMS network.

Still another aspect of the disclosure is directed to a communication system comprising: a detection device having a first LED emitter wherein the first LED emitter is configurable to emit a first target wavelength during a trough or a peak determined by an ECG R-wave, a second LED emitter wherein the second LED emitter is configurable to emit a second target wavelength during the trough or peak determined by the ECG R-wave, a detector optically isolated from the first LED emitter and the second LED emitter, a detection device processor configured to receive an input from the detector; a power supply in communication with the detection device to power the detection device; a server computer system; a measurement module on the server computer system for permitting a transmission of a measurement from a system for detecting physiological characteristics over a network; and at least one of an API engine connected to the detection device to create an message about the measurement and transmit the message over an API integrated network to a recipient having a predetermined recipient user name, an SMS engine connected to the detection device to create an SMS message about the measurement and transmit the SMS message over a network to a recipient device having a predetermined measurement recipient telephone number, and an email engine connected to the detection device to create an email message about the measurement and transmit the email message over the network to a recipient email having a predetermined recipient email address. A storing module can also be provided on the server computer system for storing the measurement on a detection device server database. Additionally, the detection device can be connectable to the server computer system over at least one of a mobile phone network or an Internet network, and a browser on a measurement recipient electronic device is used to retrieve an interface on the server computer system. A measurement recipient electronic device is connectable to the server computer system over a cellular phone network. Additionally, the measurement recipient electronic device can be a mobile device.

### INCORPORATION BY REFERENCE

All publications, patents, and patent applications mentioned in this specification are herein incorporated by reference to the same extent as if each individual publication, patent, or patent application was specifically and individually indicated to be incorporated by reference. References include, for example: U.S. Pat. No. 7,738,935 B1 to Turcott, issued Jun. 15, 2010, for “Methods and Devices for Reduction of Motion-Induced Noise in Pulse Oximetry;” U.S. Pat. No. 8,073,516 B2 to Scharf issued Dec. 6, 2011, for “Separating Motion from Cardiac Signals Using Second Order Derivative of the Photo-Plethysmogram and Fast Fourier Transforms;” U.S. Pat. No. 6,801,799 B2 to Mendelson, issued Oct. 5, 2004, for “Pulse Oximeter and Method of Operation;” U.S. Pat. No. 8,346,327 B2 to Campbell, issued Jan. 1, 2013, for “Method for Identification of Sensor Site by Local Skin Spectrum Data;” U.S. Pat. No. 8,133,176 B2 to Porges, issued Mar. 13, 2012, for “Method and Circuit for Indicating Quality and Accuracy of Physiological Measurements;” and U.S. Pat. No. 7,691,067 B2 to Westbrook, issued Apr. 6, 2010, for “Method for Measuring Central Venous Pressure or Respiratory Effort;”

US Publications: US 2010/0324390 A1 to McLaughlin, published Dec. 23, 2010, for “Measurement of Oxygen Saturation of Blood Haemoglobin,” US 2013/0317331 A1 to Bechtel, published Nov. 28, 2013, for “Monte Carlo and Iterative Methods for Determination of Tissue Oxygen Saturation;” US 2015/0057511 A1 to Basu, published Feb. 26, 2015, for “Sensor and Method for Continuous Health Monitoring;” US 2015/0011854 A1 to Frix, published Jan. 8, 2015, for “Continuous Transdermal Monitoring System and Method;” US 2013/0303921 A1 to Chu, published Nov. 14, 2013, for “System and Method for Measurement of Physiological Data with Light Modulation;” and US 2014/0275888 A1 to Wegerich published Sep. 18, 2014 for “Wearable Wireless Multisensor Health Monitor with Heat Photoplethysmograph;” and

References: Fontaine et al. “Reflectance-Based Pulse Oximeter for the Chest and Wrist” Worcester Polytechnic Institute (2013); Pujary, “Investigation of Photodetector Optimization in Reducing Power Consumption by a Noninvasive Pulse Oximeter Sensor,” Worcester Polytechnic Institute (2004); Haahr, “A Novel Photodiode for Reflectance Pulse Oximetry in Low-Power Applications,” Proceedings of the 29th Annual International Conference of the IEEE EMBS (August 2007); and Duun, et al. “A Ring Shaped Photodiode Designed for Use in a Reflectance Pulse Oximetry Sensor in Wireless Health Monitoring Applications,” IEEE Sensors Journal, Vol. 10, No. 2 (February 2010).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an external view of a physiologic sensor device according to the disclosure;

FIG. 2 illustrates the underside of the sensor device of FIG. 1;

FIG. 3 illustrates the internal electronic components of the sensor device of FIG. 1;

FIG. 4 illustrates a cut-away view of the internal components of the sensor device of FIG. 1;

FIGS. 5A-B illustrates reflectance recording of red and infrared light during an induced hypoxia episode;

FIG. 6 illustrates a physiologic sensor according to the disclosure;

FIG. 7 illustrates a physiologic sensor according to the disclosure suitable to wear around a limb of a user;

FIG. 8 illustrates a physiologic sensor according to the disclosure;

FIG. 9A illustrates the opto-electronic response of skin to normal oxygen levels at 660 nm illumination;

FIG. 9B depicts the photonic influence of a less than normal level of oxygen in the illuminated skin tissue;

FIG. 10A illustrates the photonic response of skin to normal oxygen levels at about 805 nm illumination;

FIG. 10B illustrates the photonic response of skin to decreased oxygen levels at about 805 nm illumination;

FIG. 10C illustrates five graphic displays of the cardiac cycle-related phenomena relating to the timing of LED power and photocurrent signal sampling according to the disclosure compared to a trough and peak timing interval; and

FIG. 11 is a flow diagram of the false alarm-prevention and real alarm detection during sensor motion algorithm.

### DETAILED DESCRIPTION

### I. Devices

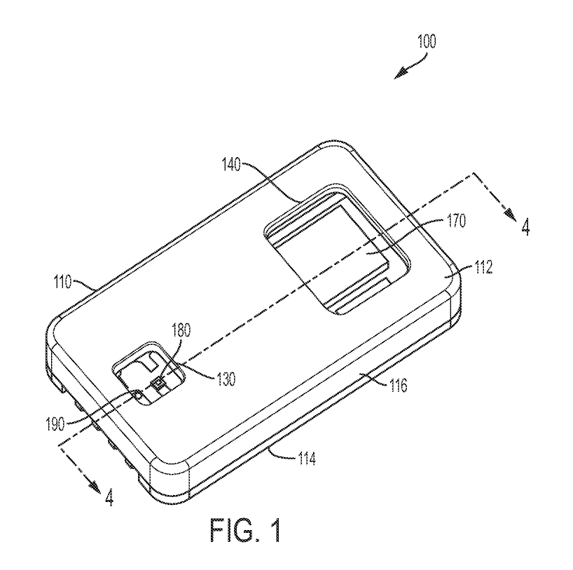

FIG. 1 illustrates an external view of a physiologic sensor 100 such as a reflectance mode oximeter sensor. The physiologic sensor 100, has a housing 110 which is configurable to enclose the internal circuit components. The housing 110 can have a top side 112, a bottom side 114 and four side walls 116. The housing can be made from separate components, or as a single unit. The housing can take on a variety of shapes. For example, the housing can be square, rectangular, round or ovoid in a two dimensional plane. As depicted in FIG. 1, the housing is rectangular. The housing 110 can be formed from any suitable material, including, for example, plastic and metal. The housing 110 has a first housing aperture 130 and a second housing aperture 140. The openings can form a recess from the top planar surface of the housing 110. A power supply (not shown) can also be contained within housing 110. Suitable power supplies include, for example, a lithium ion battery. However, as will be appreciated by those skilled in the art, power can be delivered to the sensor by any suitable means including, for example, delivery via the power and ground solder pads on the bottom of the sensor circuit board.

The physiologic sensor 100 has a first LED 180 emitting a first center wavelength of 640 nm to 680 nm, more preferably about 650 nm to 670 nm, and even more preferably about 660 nm and a second LED 190 emitting a second center wavelength of 790 nm to 830 nm, more preferably about 800 nm to 820 nm, and even more preferably about 805 nm (first LED 180, and second LED 190). The first LED 180, and the second LED 190 are positioned such that light from the LED may pass through a first internal space filled with optically clear epoxy encapsulation and exit through a first housing aperture 130 to be applied to and/or passed through the skin of a subject. A second housing aperture 140 admits the diffused and reflected LED light from the subject's skin through a clear epoxy encapsulation into a second internal space housing a silicon photodiode 170. The silicon photodiode 170 has a spectral sensitivity profile that rises between about 805 nm and 1000 nm.

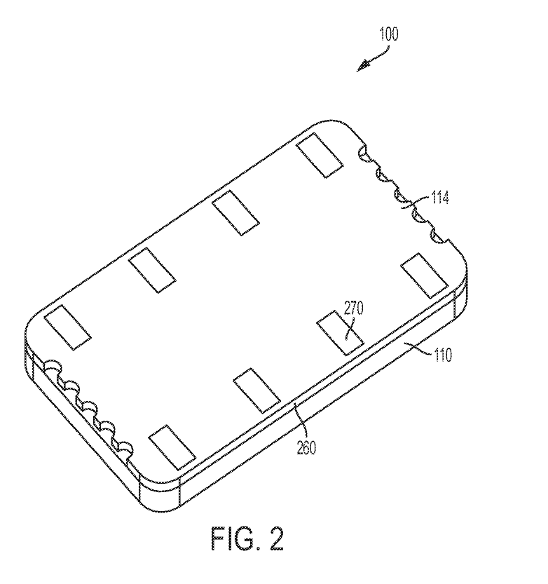

FIG. 2 shows the inward facing surface of the circuit board 114 of the physiologic sensor 100, which is attachable to the bottom face of housing 110 (i.e., the surface of the circuit board that faces into interior of the housing. A two- or more layer electronic circuit substrate 260 has one or more interface electrical connection solder pads 270 positioned for mounting the sensor on a supporting electronic circuit and electrical power supply and mechanism, comprising the remainder of the sensor interface to the subject.

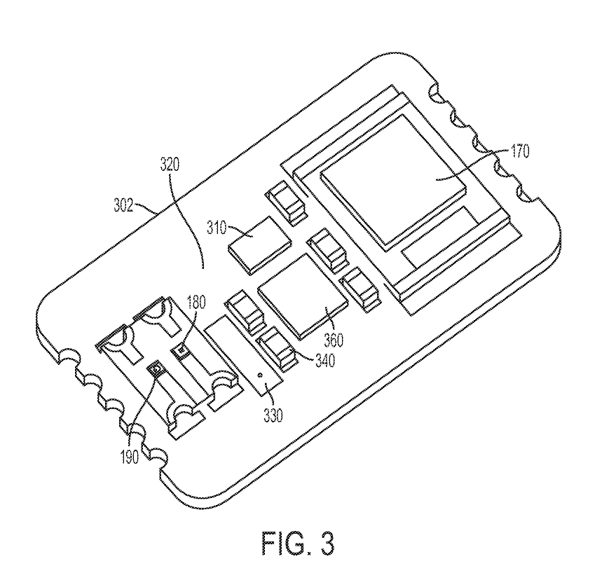

FIG. 3 shows an exemplar layout of internal components of the physiologic sensor 100 of FIGS. 1 and 2. A first LED element 180, and a second LED element 190, are mounted, for example, in a pre-assembled module to the circuit substrate 302 using conventional surface-mount technology (“SMT”) assembly techniques to generate the light output of the sensor. Circuit traces (not shown) connect each of the two LEDs to a respective drive power transistor 310. The LED power traces are covered by a layer of circuit mask 320 to electrically isolate the power traces from an internal surface of the housing 110. An electrical contact pad 330 is positioned to be bonded with electrically conductive adhesive to the underside of the internal wall of the housing 110. The electrical contact pad 330 is connectable to an AC coupling capacitor 340, which, in turn is connectable to an inverting input pad of one of two operational amplifiers on a circuit 360, such as a dual op amp chip, for detection and amplification of the ECG signal of the sensor. A second operational amplifier on the chip, or alternatively using separate transistors in ‘current mirror’ circuit layout, is configurable to receive an output signal of photodiode 170 and performs impedance transformation and amplification of the sensor photocurrent output signal.

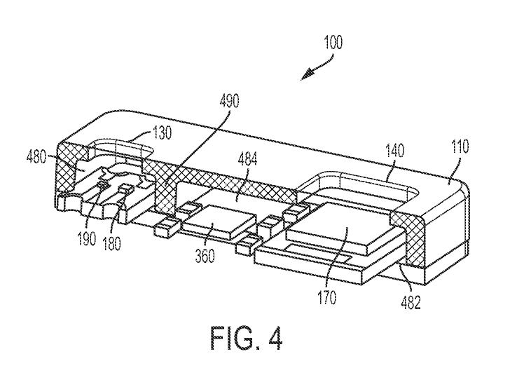

FIG. 4 shows a sectional view along the lines 4-4 of FIG. 1 of the physiologic sensor 100 revealing an orientation of the internal components positioned within the housing 110 to each other and to two optical windows that are formed by a first housing aperture 130, and a second housing aperture 140. A first internal space 480, accessible by a first housing aperture 130, containing the two LEDs (first LED 180, and second LED 190), is fillable at assembly with a suitable optically clear epoxy compound. Suitable compounds include, for example, EPO-TEK 301-2 (available from Epoxy Technology, Inc., Billerica, Mass.). An internal barrier or wall 490 of the housing 110 is positioned between, for example, the first internal space 480 and the second internal space 482. The wall 490 can block LED light from traveling within the housing from the first LED 180, and the second LED 190 to a photodetector such as silicon photodiode 170. The wall 490 can be formed from any suitable visible- and IR-opaque material, including, for example, metal. Additionally, the wall 490 can provide structural support to the housing 110. An ECG R-wave pre-amplifier circuit (not shown) can, in some configurations, be positioned within the second internal space 482 adjacent the photodiode 370. A space 484 adjacent the circuit 360 can also be filled with a suitable optically clear epoxy compound. As will be appreciated by those skilled in the art, space 484 can be continuous with space 482. Alternatively, the circuit components can be positioned in the space adjacent to the LEDs 180, 190 and the barrier 490 adjacent to the photodiode 170. Each space 480, 482, 484 can be filled with an optically clear epoxy compound to form separate optical paths and to protect the internal circuit components.

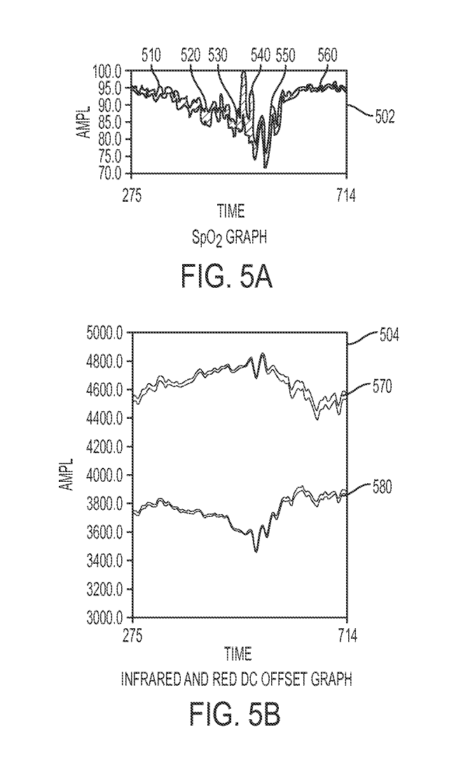

FIG. 5A is a screen shot that illustrates calculated SpO₂ and DC offset per unit time (input data values vs. seconds). The first data display 502 illustrates results starting while the subject was breathing room air. At point 510, nitrogen gas was added to the subject's breathing gas, resulting in declining SpO₂ 520. Episodes of sensor motion artifact 530 resulted in erroneously wide swings of the calculated SpO₂ graph 540. After reducing the subject's SpO₂ to about 70% by breathing room air mixed with nitrogen gas, the subject was returned again to breathing room air 550 and was shortly thereafter again at SpO₂ levels over 90% 560. In FIG. 5B a second data display 504 illustrates two double line traces consisting of the “trough” and “peak” numerical data values of reflected infrared light 570 and red light 580 used to calculate the SpO₂ values plotted in the upper graph.

During the period when the subject had normal calculated SpO₂ values, the infrared and red “trough” and “peak” traces are seen to move in tandem as the respective values were affected by sensor motion. Then, as subcutaneous tissue oxygen saturation begins to decline, these traces diverge, with the red peak and trough traces decreasing and the infrared traces increasing in value. Even with more pronounced sensor motion-induced signal artifacts, these traces continue to move in tandem; even when the motion artifacts were so severe as to compromise the calculation of SpO₂ data. Despite this loss of accurate SpO₂ data output, the DC offset remained available as a secondary indicator of oxygenation alarm status. There were several disturbances in the initially normal SpO₂ data, where, under previous methods, a false alarm may have been generated until the motion artifacts stopped. On the other hand, during the middle and latter part of the test, intermittent, valid SpO₂ data revealed true hypoxemia, which should generate an alarm response. The corresponding DC offset value, through the same time course, could have validated both the “normal-SpO₂-with-motion artifact” status and the “true hypoxia” condition alarm status, despite the intermittent loss of accurate SpO₂ data. When the nitrogen dilution of the breathing gas is discontinued, and the subject's oxygen saturation returns to normal, the numerical offset between the red and infrared DC signals returns to its previously normal-saturation DC offset.

Monitoring with the device during extreme physical exertion to near exhaustion should display a unique and potentially useful phenomenon with regard to the relationship between SpO₂ and DC offset. It is anticipated that a subject will be able to maintain, with increasing breathing frequency and depth, a nearly constant and normal SpO₂ during exercise. It is further expected that exercise at elevated altitude will consistently decrease the SpO₂ value throughout the exercise episode. However, as the exercising subject nears maximum work output, and approaches exhaustion, the physiologic control of blood flow to muscles will likely override the demand for perfusion of the skin; resulting in a photonically detectable change in subcutaneous tissue metabolism from aerobic toward anaerobic. With the device, this transition is anticipated to take the form of increasing reflectance “DC offset” in the presence of a “normal” or only slightly decreased SpO₂ value compared with the prior non-exertion value. The device uniquely presents the opportunity to further characterize this phenomenon and to evaluate its potential as an aid to physiologic monitoring of physical training and extreme performance.

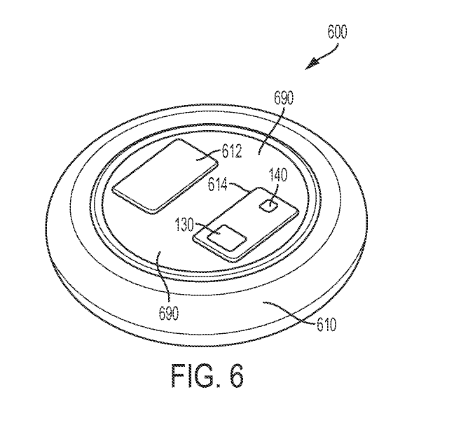

FIG. 6 depicts another configuration of a sensor device 600 with a housing 610 that is adapted for wireless vital signs monitor for use with healthy newborn infants during their transition period from birth to 24-48 hours of age. The sensor's LED window, first housing aperture 130, and photodiode window, second housing aperture 140, provide light paths through the sensor cover 614. Surrounding the device 600 are additional bio-sensor interfaces 690 and a second skin contact electrode 612, which, along with the housing 610 provides the input signal for the infant monitor's ECG R-wave pre-amplifier circuit within the sensor device 600. The above components, and the supporting battery power supply, signal processing, and wireless communications circuits are further contained within a hermetically sealed and cushioned electronic housing 610.

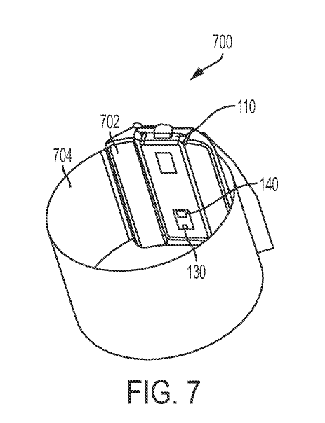

FIG. 7 is a further embodiment of a sensor device 700 suitable for physiologic monitoring of children and adults in either a clinical or non-clinical setting (e.g., medical or non-medical application). In this configuration, monitor module is designed to be applied, with the housing 110 in contact with the skin, to the upper arm and held in place with a retaining arm band 704 and buckle 702. This embodiment places the housing 110, with its LED window 130 and photodiode window 140 in contact with the skin of a subject. This module is configurable to connect wirelessly to a signal processing, data display, and battery power module (not shown). Graphic display and user interface can be implemented with a “Smart-enabled” personal device, such as an iPhone, iPad, etc. or other device, which could be positioned at the subject's wrist, on the handlebars of a bicycle, or mounted to eyeglasses for convenient viewing. This embodiment allows an exercising subject to have free, unimpeded use of hands and arms without compromising signal detection or processing of the device.

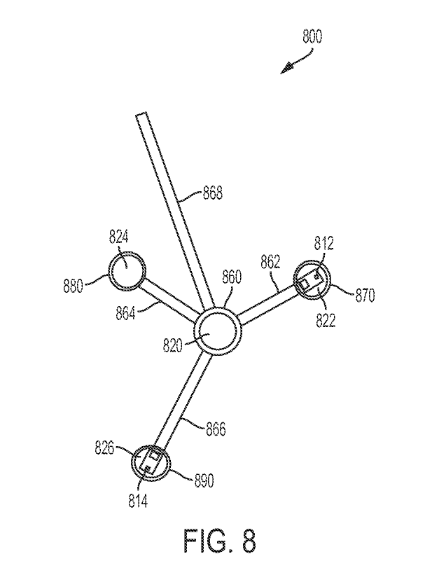

FIG. 8 depicts a further embodiment of a sensor device 800 according to the disclosure. The sensor device 800 is configurable as part of an integrated, multisensor harness for physiologic monitoring of intensive care newborn infant patients. Four skin contact pads 820, 822, 824, 826 are designed for attachment to, for example, the mid-chest 860, right upper chest 870, left upper chest 880, and left lower abdomen 890, respectively. These skin contact pads 820, 822, 824, 826 are interconnectable with suitable connectors such as straps 862, 864, 866 having embedded electronic conductors. The harness electronically interfaces with the physiologic monitor signal processing and display system via a main conductor strap 868 that passes, for example, over the left shoulder of an infant. The first sensor device 812 in the skin contact pad 820 that is configured to engage a right upper chest 870 is configurable to detect “pre-ductal” oxygenation status, while a second sensor device 814 in the skin contact pad 826 that is configured to engage a lower left abdomen 890 is configurable to detect the “post-ductal” oxygenation status of the infant. Each of the first sensor 812, and the second sensor 814 can engage in sensing simultaneously. Additionally, each of the four skin contact pads 820, 822, 824, 826 attach to the infant by, for example, a replaceable hydrogel adhesive, such as Promeon Hydrogel 027 (available from R&D Medical Products, Lake Forest, Calif.) patch, providing four electronic contacts with the skin of the infant for typical ECG and breathing monitoring functions as well as planned new monitoring modes.

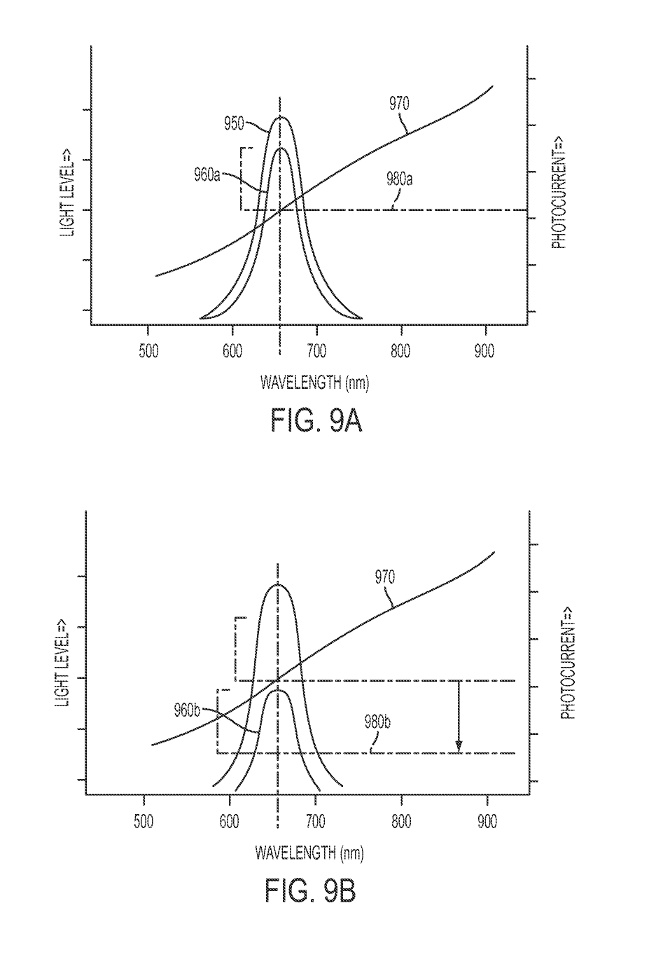

FIG. 9A graphically portrays the opto-electronic response of the device to illumination of the skin by an LED with a center wavelength of about 660 nm. The spectral emission profile of the LED 950 about its 660 nm center wavelength, is displayed relative to the spectral response curve 970 of the silicon photodiode. When illuminating normally oxygen-supplied skin, the return light level 960a is somewhat attenuated by Raleigh scattering and absorption by oxygen saturated blood hemoglobin and tissue, but has not been changed in center wavelength. The photocurrent level 980a produced is a function of the center wavelength of the returned light interacting with the photodiode at about 660 nm.

FIG. 9B depicts the photonic influence of a less than normal level of oxygen in the illuminated skin tissue. Reduced hemoglobin absorbs more of the 660 nm center wavelength light in addition to the Raleigh scattering and absorption by the illuminated tissue. Because there is no re-emission of 660 nm photons at shorter or longer wavelengths associated with anaerobic metabolism in the skin, the resulting attenuation of the light level received at the photodiode results in a lower level of photocurrent 980b produced.

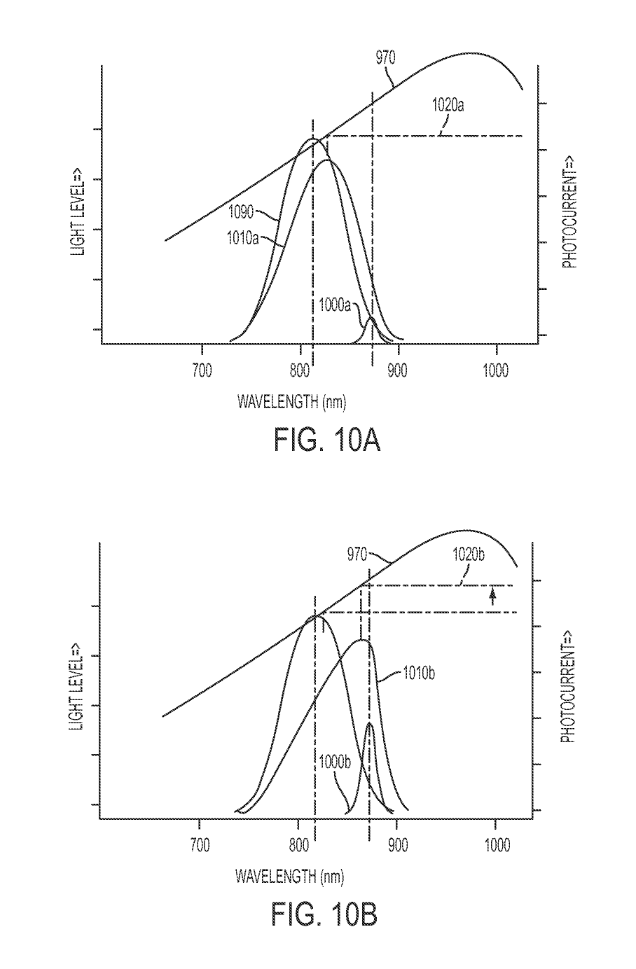

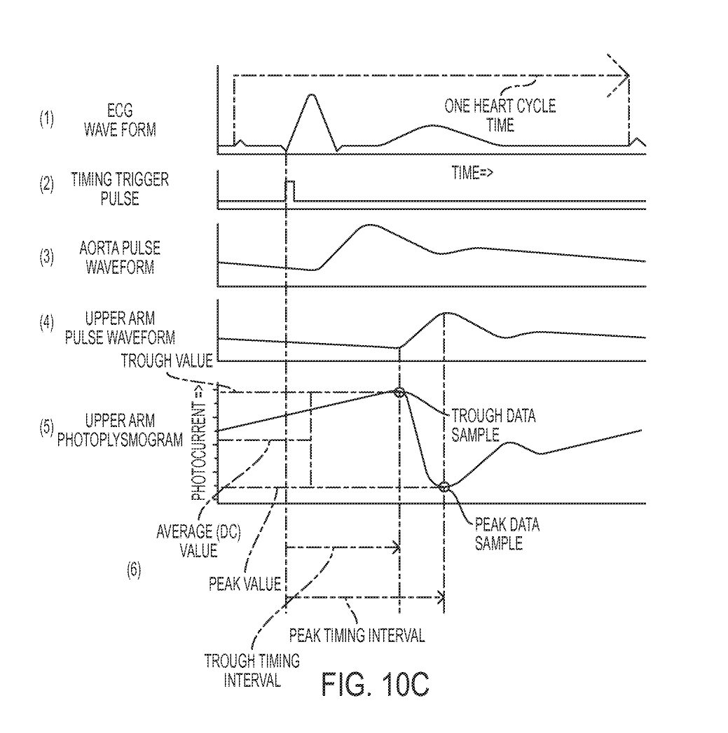

FIG. 10A depicts a photonic response of skin tissue to light having a second center wavelength emission in the range of 800 to 820 nm. Under normal levels of oxygen in the tissue, the majority photonic effect is Raleigh scattering, as with 660 nm light. However, the normally-present very low levels of pyruvate, lactate, and reduced nicotinamide adenine dinucleotide (“NADH”) present in the skin absorb photons at about 810 nm and re-emit only a small amount of photons at a lower energy level, or longer wavelength 1000a. This minor degree of Stokes' shift effect 1010a is normally not noticeable relative to the majority Raleigh-scattered returned light at the photodiode. However, as oxygen becomes less available to the illuminated skin tissue, the rate of anaerobic glycolysis increases and pyruvate, lactate and reduced NADH accumulate within the cells and diffuse into the interstitial fluid. As shown in FIG. 10B, the increase in the level of molecular products of anaerobic metabolism in the illuminated skin results in a larger proportion of the returned light being Stokes′-shifted to a longer wavelength 1000b. The combination of Raleigh scattered and increased Stokes' shifted light increases the center wavelength value of the return light. The steeply upward ramping spectral sensitivity profile of the silicon photodiode detects this upward-shifted spectral peak 1010b, i.e. the combination of Raleigh scattered and the increased level of Stokes' shifted light, and produces an increased level of photocurrent 1020b.

FIG. 10C presents five graphs (1)-(5) covering the time period of one heart cycle with reference to peak and trough timing shown in FIG. 10C(6). FIG. 10C(1) depicts the electrocardiogram, having the recognized sequence of P through T waves. The QRS complex is unique in its content of high frequency signal and this quality allows precise detection of the onset of the R-wave portion.

FIG. 10C(2) depicts the electronic logic signal pulse derived from the leading edge of the R-wave. It is this trigger pulse that is used to start timing periods, which end in the “trough” and “peak,” respectively of the PPG signal.

FIG. 10C(3) depicts the pressure waveform of the blood within the aorta, immediately past the aortic valve. This pressure wave begins its rapid upward transition as the aortic valve opens. A slight dip, then rise in pressure, called the dicrotic notch, occurs during the declining portion of the aortic pressure wave, due to the closing of the aortic valve at the end of the heart pumping action. Being able to recognize this feature is commonly considered an indication of a high quality signal, be it from an in-line blood pressure sensor, or the photoplethysmograph of a pulse oximeter.

FIG. 10C(4) depicts the blood pressure waveform that would be recorded in a peripheral artery, such as in the upper arm or wrist. It differs from the aortic pressure waveform in the timing of its peak and in its lower amplitude.

FIG. 10C(5) depicts the photocurrent waveform received from a pulse oximeter sensor. As each heart cycle occurs, the arteriolar blood volume wave that flows through the illuminated skin variably absorbs the light. This results in a decreasing, or downward-going waveform of photocurrent with each pulse wave of blood. However, because medical personnel are most familiar with an upward-going pulse wave, as with manual palpation of the pulse at the wrist, or the in-line arterial blood pressure waveform of an intra-arterial pressure monitor, the pulse oximeter waveform is commonly inverted, electronically or in software, to be displayed in the more familiar upward-going format. Thus, as stated, the raw signal of a PPG is a downward waveform of photocurrent, as depicted. The two points on this waveform that are recorded with each heart cycle and used to calculate the SpO2 occur at the “trough” (as it would be referred to in a pressure waveform, such as in FIG. 10C(4) and the following “peak” inflection. See, FIG. 10C(6) depicts the correlation of the R-wave timing trigger pulse to the “trough” and “peak” inflections of the PPG; defining the “trough” timing interval and the “peak” timing interval. Pulse oximeters record the entire plethysmogram waveform with a rapid series of alternating red and infrared illuminations of the skin to obtain about 200 data samples at each wavelength per second. This continuous digital data stream allows for graphic display of the full PPG waveform on the monitor screen, but the actual signal-derived data used for calculation of SpO2 is obtained only at the two brief periods of the waveform at the inflection points. The present invention uses the R-wave-derived timing pulse to record the characteristic time intervals to these inflection points during the initialization of the system at the beginning of each application; then times the data sampling accordingly. By this means, the LEDs need to be powered for only two very brief periods of the heart cycle to obtain fully valid data.

### II. Operation

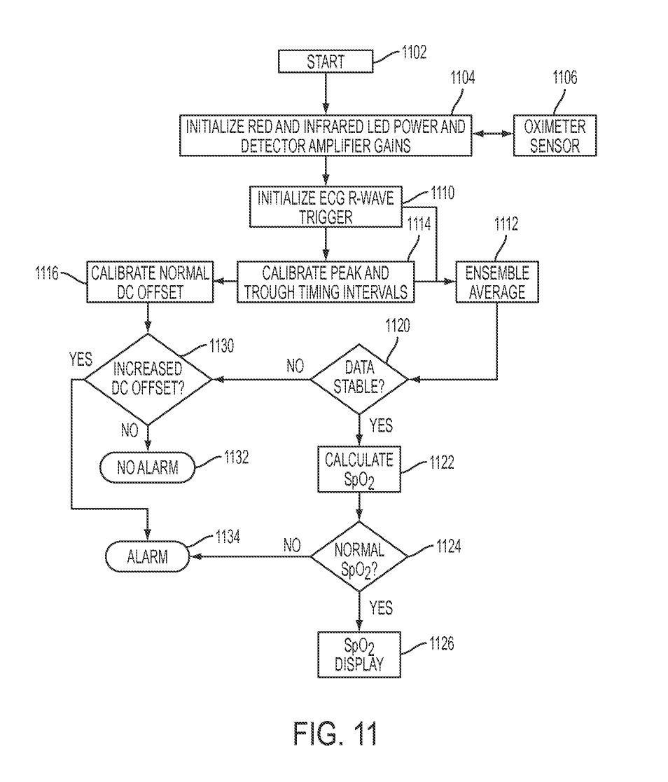

FIG. 11 is a high level flow diagram of the operation of the sensor device reflectance oximeter. Operation starts 1102 and is followed by an initializing of red and infrared LED power levels and the detector amplifier gain 1104 to be applied during the illumination of each LED. Once initialized, the oximeter sensor is activated 1106 in full photo PPG mode, with detection of waveform maxima and minima, i.e. “troughs” and “peaks” being confirmed. Thereafter the ECG R-wave-derived sample timing trigger pulse is created 1110. The system then measures the average “trough” and “peak” timing intervals 1114 relative to the preceding R-wave-derived timing trigger pulse. Following the ECG R-wave trigger activation 1106 and the calibration of “trough” and “peak” timing intervals 1114, the system begins to obtain and ensemble average, for instance 5 cardiac cycles of the signal values 1112 that occur at the predetermined timing intervals relative to the ECG R-wave-derived trigger pulses. The ensemble averaged data is then reviewed to determine if the data produced is stable 1120. If the ensemble averaged data is stable, calculation of SpO₂ is performed performed and the resulting value is displayed. If the calculated SpO₂ value is within the “normal” range, no alarm is issued. If the SpO₂ value is outside of the “normal” range, an alarm is issued.

Additionally, following the calibration of “trough” and “peak” timing intervals step 1114, the system can calibrate a normal DC offset 1116. Once the normal DC offset, i.e. correlating with a “normal” range SpO₂ value, is calibrated 1116. With the DC offset initialized at about 10 counts and stable for a period of time, the LED power and signal amplifier values are “locked” in system memory and are used throughout the current sensor application. If the data becomes unstable 1120, then the system determines whether the DC offset is increased 1130 from the previous “normal” range level. If the DC offset is not increased, then no alarm needs to be activated 1132. If the DC offset is increased 1130, then an alarm is activated 1134. If the data is stable 1120, then the system calculates the SpO₂ of the patient 1122. If the SpO₂ is normal 1124, then the SpO₂ is displayed 1126. If the SpO₂ is not normal 1124, then an alarm is activated 1134.

Operation of the process of FIG. 11, in more detail, starts with an initialization of the LED power, detector gain, and the heart cycle trigger signal processing system. During this process, two objects can be achieved. First, both the red and the infrared emitter power supplies can be step-wise adjusted to maximize the resulting reflected light value after the detected and amplified signal at each wavelength is digitized. An upper limit adjustment for each wavelength is such that the digitized signal value can remain just beneath a maximum count of the analog-to-digital (“A/D”) converter, i.e. over 90%, but not exceeding 95% of the maximum count of the converter. Emitter power can be maximized, to help minimize signal noise, with the remaining increase achieved by adjusting the amplification of the detector signal. With the emitter power and detector signal amplifier initialization adjustments achieved for each wavelength, the resulting reflectance DC values are maximized and are, numerically, very nearly the same, with the DC infrared signal being about 10 counts greater than the DC red signal corresponding approximately to a “normal range” of SpO₂ values.

Upon completion of the initialization, the emitters can be illuminated in a continuous, repeating cycle, comprising four segments: red on and infrared off, both off, red off and infrared on, and, finally both off. This cycle repeats about 200 times per second, according to the internal clock and default software timer values of the processor circuit and program, respectively. Maximum and minimum signal values are sought from the resulting stream of amplified and A/D converted detector data. Specifically, the time intervals from the separately obtained heart cycle R-wave trigger signal to the “trough” and to the “peak” inflections of the PPG are measured. These two time interval values are then averaged over several heart cycles and the average time interval values stored in system memory. These timing interval values are then used to regulate the on-going data acquisition of the system with the present placement of the probe.

With the completion of the initialization sequence, either of two operating modes is made active. In the normal reflectance oximeter operation, each cardiac cycle generates four sampled and calculated data values: Red AC, Red DC, Infrared AC, and Infrared DC. These data are acquired by sampling red, infrared, and ambient light values at the trough and the peak time intervals from the R-wave trigger for each heart cycle. Each light value is, in turn, the average of two or more samples of oximeter emitter illumination. The ambient light value is subtracted from the red and the infrared values prior to the latter values used in calculating the AC and DC data.

The four data derived from each cardiac cycle are then ensemble averaged over five to six cardiac cycles to reduce low-frequency variation that occurs due to breathing. The resulting averaged values are then used to compute and display a current SpO₂ output value for each heart cycle. Heart rate is also computed from the time interval between cardiac cycle R-wave trigger signals and displayed with the SpO₂ value.

The system then derives a relationship between the baseline DC offset and the calculated SpO₂ based on cardiac cycle-timed “trough” and “peak” data values. Assuming the initial computed SpO₂ value is in the “normal” range, the baseline DC offset is related in system memory to this “normal” value. If the initially calculated SpO₂ is below or above the “normal” range, the system will re-initialize emitter output and amplifier gain when the calculated SpO₂ value moves to the normal range.

In the event of a large sensor motion-induced disturbance of data, the system switches to acquiring reflectance light samples according to a software timer, e.g., once per second. Since the pulsatile, or AC, variation of the PPG is typically only 0.5% to 3.5% of the total reflectance light signal, the slight error introduced by this method of sampling is insignificant and the resulting “random” values very closely approximate the cardiac cycle-timed DC values for both red and infrared. Red and infrared reflectance DC values derived from either the cardiac-timed or clock-timed samples are subtracted to provide an ongoing DC offset value. Placing the desired normal range of SpO₂ in the lower region of the DC offset range allows the DC offset value to be used to bridge gaps in suitable AC signal and avoid unnecessary alarms. Both the calculated value of the SpO₂ and the DC offset are used to logically define alarm conditions. The far more robust DC offset provides a motion-tolerant means of preventing false alarms. Conversely, since the DC offset value can be obtained even in the absence of stable input signal, true tissue hypoxia can be detected at any time and the alarm issued appropriately.

When first applied to the skin, the sensor device control runs an initial auto-ranging protocol. During this process, the signal levels at both LED wavelengths are adjusted, in incremental steps of emitter power and detector amplifier gain, to maximize the resolution of analog to digital (“A/D”) conversion. This optimization is achieved when the two LED power levels and their corresponding light detector amplifier gain, produces averaged, reflected DC numerical values approaching, but not exceeding, the maximum count limit of the A/D converter; and the DC reflectance value of the 790 to 820 nm, more preferably 800 to 810 nm, and even more preferably at a center wavelength of about 805 nm LED light exceeds the DC reflectance value of the LED, which emits light at from 640 nm to 680 nm, more preferably about 650 nm to 670 nm, and even more preferably at a center wavelength of about 660 nm, by about 10 digital counts. This initialization process thus optimizes the resolution and accuracy of the computed oximetry value for each subject, and for each sensor placement location, and helps compensate for differences in skin thickness and pigmentation. It also pre-sets the DC offset value at a low, positive count to maximize its sensitivity to changes in the respective signals due to increased anaerobic metabolism in the skin.

The integrated ECG detection and analysis system is then initialized to produce a timing logic pulse corresponding with the leading edge of the user's ECG R-wave. This logic timing pulse is then used to compute the subject's heart rate and an index of the variation of the patient's beat-to-beat heart cycle timing.

A continuous PPG signal is then generated and analyzed to determine the time intervals between the ECG R-wave timing logic pulse and the immediately following “trough” and “peak” inflections of the PPG. Each application site, and each subject being monitored, will likely have unique time delays between the derived R-wave trigger pulse and the following “trough” and “peak” waveform inflections in the optical signal. This variability in time intervals is due to the unique length and elasticity of the pulse conduction pathway (arterial blood vessels) between each subject's heart and the oximeter application site.

Once the automated initialization adjustments have been completed, the LED power levels, the detector amplifier gain levels, and the “trough” and “peak” sample timing intervals are recorded and “locked” as customized parameters in the control software. Thereafter, the SpO₂ value is acquired and computed by running ensemble averaging of “trough” and “peak” signal samples including about 5 cardiac cycles. The numerical difference between the individually averaged (i.e. (“trough”+“peak”)/2) values, or DC levels, is also continuously recorded as the “DC offset.”

An additional safety feature can be enabled by analysis using the continuously recorded DC offset. In the event that valid oximeter ensemble-averaged SpO₂ data cannot be obtained, such as commonly occurs when a monitored infant is crying or an adult subject coughs or moves vigorously; the reflectance DC light levels can be sampled on a default-timed basis. Obtaining reflectance light levels on a clock-timed basis, such as once per second, will provide fully adequate data for the DC offset analysis and appropriate alarm generation. Further, in situations where heart rate and oximetry values have stabilized, as during quiet sleep, the default-timed mode, using longer sampling intervals up to several seconds, may also be used as a significant sensor power conservation option with battery powered formats. At any time the intermittently sampled DC offset deviates from the “normal” range determined during initialization to represent significant decrease or abnormal increase in SpO₂, the regular operation of the oximeter would immediately resume. If a DC offset alarm condition were found to exist, with or without a valid computed SpO₂ value, an alarm output would be generated and the system would continue to attempt to calculate a valid SpO₂ value.

Power consumption in portable, battery powered oximeters may be significantly reduced by the device's use of cardiac cycle-timed data sampling. The narrow segments of the total PPG, or AC, signal needed for computation of the SpO₂ value are acquired at two specific points in each cardiac cycle: the “trough” and the following “peak.” Once the system is initialized and the “trough” and “peak” sampling is occurring according to the timing parameters, LED power can be left off during all other portions of the heartbeat cycle. Thus, LED power usage may be reduced to less than 5% duty cycle with continuous acquisition of fully valid trend oximetry values. LED power could be reduced even further, to less than 1% duty cycle, in the default timed sampling mode where only the two DC reflectance levels are acquired at clock-timed intervals. These power conservation means allow use of a smaller battery and/or longer periods of use between battery recharges, without degrading the value of the data, or the security and clinical validity of the alarm system.

In locked mode, the sensor system detects four timed LED-illuminated reflectance intensity values for each heart cycle. Each of these four light intensity samples is an average of at least two oximeter illumination sample cycles, each starting at the respective defined sampling time, according to the heart cycle timing parameter value. Averaging of multiple samples during each oximeter illumination cycle to produce each output value reduces aliasing from ambient lighting and from other sources of ambient optical and electromagnetic interference (EMI) noise.

The resulting acquired data for each heart cycle, in sequence, include: (a) 660 nm “trough,” (b) about 805 nm “trough,” (c) 660 nm “peak,” and (d) about 805 nm “peak.” Time-adjacent, corresponding ambient light (both LEDs off) detection samples are subtracted from each of the LED-illuminated signal samples prior to further computation. As these signal values are acquired, they are each further processed through about five heart cycles of running ensemble averaging to help remove low frequency variations, such as the variations that are normally associated with breathing.

Four computed intermediate values are then derived from these four basic data, as is well known in the oximetry field. A “660 nm AC value” is computed by subtracting the 660 nm “peak” value from the 660 nm “trough” value. A “660 nm DC” value is computed as the numerical average of immediately adjacent 660 “trough” and 660 nm “peak” values. “805 nm AC” and “805 nm DC” values are similarly computed using the corresponding illumination samples. The output SpO₂ is then computed, first as an “R-value.” The R-value is converted to a percent of saturation by an empirically derived formula. The R-value computation is based on the ratio-of-ratios method of detecting minimal AC variations superimposed on large DC base signals. The formulas for this extraction and conversion process are well known to the oximetry art as:

R-value=(660 nm AC/660 nm DC)/(805 nm AC/805 nm DC); and

SpO₂=−25(R-value)+110.

The device system further analyzes the two derived DC values as to their running average subtraction, or numerical offset, or DC offset, from each other. With the light emitter and detector amplifier controls initialized and locked, as described above, the DC illumination values generated by the two emitters tend to be very nearly the same converted value and vary closely in tandem as long as oxygen saturation of the monitored tissue is stable. Tandem variation in the reflectance DC values is primarily due to changes in the contact pressure with which the reflectance oximeter sensor presses the skin area being analyzed. Greater pressure reduces the amount of mainly low-pressure capillary and venous blood in the tissue, affecting both the first wavelength emission and the second wavelength emission reflectance signals nearly equally. Further, with fewer, or no, cycles of ensemble averaging, the reflectance DC tandem variation also reveals breathing-induced “artifact.”

As long as the skin tissue oxygen availability status remains stable, the reflectance DC offset value remains small and stable within an empirically defined range; i.e. corresponding approximately to a range of SpO₂. Unique to the device, this reflectance DC offset value is used as a secondary, approximate index of oxygen availability in the tissue being monitored. It is to be expected that the subject being monitored could frequently and randomly move in a manner that will temporarily degrade the relatively tiny (0.5-3.5%) AC portions of the respective total detected photocurrent signals. One of the contributions to the art by the device is to use the reflectance DC offset value to confirm that a tissue hypoxia, or anaerobic metabolism, condition exists in the skin being illuminated prior to issuing the alarm response. In the event that the DC offset value, i.e. the numerical difference between the running averages of the 660 nm and the 805 nm reflectance DC values, has not changed significantly since a valid, accurate SpO₂ value was available and within the “normal” range, a desaturation condition warranting an alarm is very unlikely to exist. On the other hand, even if the AC portion of the oximeter signal is temporarily degraded by poor perfusion, motion of the sensor, breathing, or ambient light, the DC offset analysis will still detect a true hypoxia condition and trigger the alarm. Accurate SpO₂ value computation and display will, of course, need to wait for the signal distortion to cease and the AC portion of the oximeter signal to return to satisfactory quality. However, a valid index of the monitored subject's physiologic safety is fully maintained, and false alarms are eliminated.

### III. Communication of Sensor Data

The systems and methods according to aspects of the disclosed subject matter may utilize a variety of computer and computing systems, communications devices, networks and/or digital/logic devices for operation. Each may, in turn, be configurable to utilize a suitable computing device that can be manufactured with, loaded with and/or fetch from some storage device, and then execute, instructions that cause the computing device to perform a method according to aspects of the disclosed subject matter.

A computing device can include without limitation a mobile user device such as a mobile phone, a smart phone and a cellular phone, a personal digital assistant (“PDA”), such as a BlackBerry®, iPhone®, a tablet, a laptop and the like. In at least some configurations, a user can execute a browser application over a network, such as the Internet, to view and interact with digital content, such as screen displays. A display includes, for example, an interface that allows a visual presentation of data from a computing device. Access could be over or partially over other forms of computing and/or communications networks. A user may access a web browser, e.g., to provide access to applications and data and other content located on a website or a webpage of a website.

A suitable computing device may include a processor to perform logic and other computing operations, e.g., a stand-alone computer processing unit (“CPU”), or hard wired logic as in a microcontroller, or a combination of both, and may execute instructions according to its operating system and the instructions to perform the steps of the method, or elements of the process. The user's computing device may be part of a network of computing devices and the methods of the disclosed subject matter may be performed by different computing devices associated with the network, perhaps in different physical locations, cooperating or otherwise interacting to perform a disclosed method. For example, a user's portable computing device may run an app alone or in conjunction with a remote computing device, such as a server on the Internet. For purposes of the present application, the term “computing device” includes any and all of the above discussed logic circuitry, communications devices and digital processing capabilities or combinations of these.

Certain embodiments of the disclosed subject matter may be described for illustrative purposes as steps of a method that may be executed on a computing device executing software, and illustrated, by way of example only, as a block diagram of a process flow. Such may also be considered as a software flow chart. Such block diagrams and like operational illustrations of a method performed or the operation of a computing device and any combination of blocks in a block diagram, can illustrate, as examples, software program code/instructions that can be provided to the computing device or at least abbreviated statements of the functionalities and operations performed by the computing device in executing the instructions. Some possible alternate implementation may involve the function, functionalities and operations noted in the blocks of a block diagram occurring out of the order noted in the block diagram, including occurring simultaneously or nearly so, or in another order or not occurring at all. Aspects of the disclosed subject matter may be implemented in parallel or seriatim in hardware, firmware, software or any combination(s) of these, co-located or remotely located, at least in part, from each other, e.g., in arrays or networks of computing devices, over interconnected networks, including the Internet, and the like.

The instructions may be stored on a suitable “machine readable medium” within a computing device or in communication with or otherwise accessible to the computing device. As used in the present application a machine readable medium is a tangible storage device and the instructions are stored in a non-transitory way. At the same time, during operation, the instructions may at some times be transitory, e.g., in transit from a remote storage device to a computing device over a communication link. However, when the machine readable medium is tangible and non-transitory, the instructions will be stored, for at least some period of time, in a memory storage device, such as a random access memory (RAM), read only memory (ROM), a magnetic or optical disc storage device, or the like, arrays and/or combinations of which may form a local cache memory, e.g., residing on a processor integrated circuit, a local main memory, e.g., housed within an enclosure for a processor of a computing device, a local electronic or disc hard drive, a remote storage location connected to a local server or a remote server access over a network, or the like. When so stored, the software will constitute a “machine readable medium,” that is both tangible and stores the instructions in a non-transitory form. At a minimum, therefore, the machine readable medium storing instructions for execution on an associated computing device will be “tangible” and “non-transitory” at the time of execution of instructions by a processor of a computing device and when the instructions are being stored for subsequent access by a computing device.

Additionally, a communication system of the disclosure comprises: a sensor as disclosed; a server computer system; a measurement module on the server computer system for permitting the transmission of a measurement from a detection device over a network; at least one of an API (application program interface) engine connected to at least one of the detection device to create a message about the measurement and transmit the message over an API integrated network to a recipient having a predetermined recipient user name, an SMS (short message service) engine connected to at least one of the system for detecting physiological parameters and the detection device to create an SMS message about the measurement and transmit the SMS message over a network to a recipient device having a predetermined measurement recipient telephone number, and an email engine connected to at least one of the detection device to create an email message about the measurement and transmit the email message over the network to a recipient email having a predetermined recipient email address. Communications capabilities also include the capability to communicate and display relevant performance information to the user, and support both ANT+ and Bluetooth Smart wireless communications. A storing module on the server computer system for storing the measurement in a detection device server database can also be provided. In some system configurations, the detection device is connectable to the server computer system over at least one of a mobile phone network and an Internet network, and a browser on the measurement recipient electronic device is used to retrieve an interface on the server computer system. In still other configurations, the system further comprising: an interface on the server computer system, the interface being retrievable by an application on the mobile device. Additionally, the server computer system can be configured such that it is connectable over a cellular phone network to receive a response from the measurement recipient mobile device. The system can further comprise: a downloadable application residing on the measurement recipient mobile device, the downloadable application transmitting the response and a measurement recipient phone number ID over the cellular phone network to the server computer system, the server computer system utilizing the measurement recipient phone number ID to associate the response with the SMS measurement. Additionally, the system can be configured to comprise: a transmissions module that transmits the measurement over a network other than the cellular phone SMS network to a measurement recipient user computer system, in parallel with the measurement that is sent over the cellular phone SMS network.

### IV. Examples

The device preferably embodies an LED light generated at a center wavelength of about 640 nm to 680 nm, more preferably about 650 nm to 670 nm, and even more preferably about 660 nm, and an LED light generated at a center wavelength of about 790 nm to 820 nm, more preferably about 800 nm to 810 nm, and even more preferably about 805 nm, in an alternating sequence, with intervening periods of no generated light. By this sequence, as with prior pulse oximeter designs, the photodiode sensor can determine the light intensity returned from each skin-illumination period and the time-adjacent ambient light intensity values. The time-adjacent ambient light intensity values are subtracted from each illuminated signal value to obtain the net signal values used for computation of SpO₂. The about 805 nm wavelength was selected, instead of the typical use of about 940 nm, as it was the available LED wavelength closest to the known isosbestic spectral absorbance wavelength of reduced hemoglobin vs. oxy-hemoglobin. The wavelength range emitted by LEDs with about 805 nm center wavelength apparently also includes the wavelength/s that will photonically excite one or more of the molecular products of anaerobic metabolism, such as pyruvate, lactate, or NADH, with the resulting re-emission of longer wavelength light that is detected at higher sensitivity, such as indicated by producing a relatively higher photocurrent, by the photodiode sensor.

It has been observed from experimentation that the photodiode sensor photocurrent response to returned light also varies in the non-pulsatile, or DC, portion of the output signal in response to induced whole-body hypoxia. Decreasing arteriolar hemoglobin-oxygen saturation, i.e. dropping SpO₂ value, was observed during prototype experimentation to be associated with progressive decrease in the DC photocurrent signal during the LED illumination of the 640 nm to 680 nm, more preferably about 650 nm to 670 nm, and even more preferably about 660 nm center wavelength. This can be logically accounted for as increased absorption of 660 nm light by the more oxygen-desaturated hemoglobin in the capillaries and veins caused by more extreme oxygen extraction by the surrounding tissues.

However, progressive increase in the DC photocurrent signal was observed during about 810 nm center wavelength LED illumination during hypoxic challenge. The about 810 nm wavelength used is very near the isosbestic spectral absorbance wavelength of hemoglobin vs. oxy-hemoglobin. This means that no change in light absorbance by hemoglobin is expected to occur at this wavelength relative to the amount of oxygen bound to the hemoglobin. Rather, it is surmised that the increased photocurrent signal with deepening hypoxia is likely due to accumulation of one or more molecular products of anaerobic metabolism in the illuminated skin tissue. It is known that anaerobic glycolysis results in rising intracellular levels of pyruvate, lactate, and NADH. These molecules are also known to be “photophores” in the infrared (“IR”) portion of the spectrum; meaning that they can be excited by IR wavelength photons. This phenomenon is currently being used for non-destructive, multi-photon stimulation visible microscopy of living tissue. This technique uses a near-infrared (“NIR”) laser light source to “light-up” intracellular mitochondria that have elevated levels of lactate and NADH due to anaerobic metabolism. The use of a high intensity NIR laser source produces multi-photon stimulation of these metabolite molecules, resulting in Raman scattered re-emission of light in the higher energy, shorter wavelength, visible portion of the spectrum; thus enabling use of visible light imaging optics and image capture sensors.

It is surmised from experimental observations with an engineering prototype of the present disclosure that a lower intensity source, such as a NIR LED, would likely produce single-photon NIR excitation of these same molecules, resulting in re-emission at lower energy, longer wavelengths (i.e. Stokes' shift). The device detects this increase in wavelength-shifted re-emission due to the upward slope of the photodiode spectral response profile correlating with increasing wavelength between about 805 nm and about 1000 nm. Thus, increasing anaerobic metabolism results in increased local levels of molecular products of anaerobic metabolism, such as pyruvate, lactate and NADH, which re-emit at a longer wavelength when excited with about 805 nm light. The increased amount of longer wavelength light, mixed with the unchanged wavelength, also known as Raleigh scattered, LED wavelength light, results in shifting the combined center wavelength of the returning light toward the longer wavelength, more sensitive spectral region of the detector; thus increasing the photocurrent output of the photodiode relative to the same LED power level and resulting primary emission intensity.

The opposing variations in DC photocurrent associated with deepening hypoxia enables use of simple subtraction of the 660 nm DC signal value from the about 805 nm DC signal value, producing a difference, or “DC offset,” value, as an analog of anaerobic metabolism in the skin. Since skin tissue is known to have much lower biologic priority for oxygen delivery compared with the more vital body organs, a non-invasive skin-mounted sensor such as the device may be useful as a sensitive and reliable surrogate indicator of general body hypoxia and/or the potential for compromised oxygen delivery to vital organs.

The device uses this robust diverging DC reflectance signal phenomenon, also referred to herein as “DC offset,” to secondarily validate the more precise, but also more easily distorted, AC signal-derived oximetry value relative to the need for generating an alarm. While not enabling computation of an accurate hemoglobin/oxygen saturation value, the running difference between the two sensed DC light photocurrent values has been found to robustly indicate the general status of subcutaneous tissue oxygen metabolism. Even though motion-induced variations in reflected light levels might be pronounced, the numerical difference between the two DC light values has been found to remain very nearly the same with sensor motion-induced changes alone. However, if the level of oxygen in the skin tissue even slightly changes, the difference between the two wavelength DC reflectance levels, i.e. the DC offset value, measurably changes. Experiments have shown as much as 1000% change in the reflectance DC offset value, corresponding to a change in computed SpO₂ value from 96% down to 70%, then back up to 96%, due to inhalation of nitrogen-diluted air by an adult test subject. The device uses this phenomenon to prevent generating false alarms during periods of high sensor motion artifact, when accurate computation of hemoglobin-oxygen saturation is compromised. This backup alternative evaluation is expected to also be clinically useful as an index of metabolic stress from a wide variety of causes.

Continuous surveillance to detect this phenomenon may be found clinically useful as a secondary index of tissue aerobic vs. anaerobic energy transfer metabolism during periods of sensor motion-induced signal artifact. Common situations, such as with crying infants in the Newborn Intensive Care Unit (“NICU”), could have the safety of not missing real hypoxia conditions, despite sensor motion-induced signal noise that prevents computation of an accurate SpO₂ value.

Utility of this method is also anticipated in non-medical applications of vital function monitoring during strenuous activity, such as athletic training and competition, and during recreation and work at high altitude and in other hazardous environments, such as firefighting, aircraft piloting, space exploration, diving, combat, etc. It is known in the study of human physiology that both strenuous muscular activity and major illness will preferentially divert arterial blood flow to the brain, heart, and muscles at the expense of perfusion of less life-essential tissues, such as the gut and skin. This response is balanced against the need to dissipate excess body core heat, which is generated as a byproduct of muscle contraction. As core temperature increases, perfusion of the skin is reflexively increased to help dissipate the excess heat through radiation and convection, and through evaporation of sweat. However, at some point on the rising scale of extreme exertion, chemical and neurologic signals from muscles verging on anaerobic metabolism may override the elevated skin perfusion response to conserve perfusion to the muscles and more vital organs. This, in turn may result in a more rapid rise in core temperature to the ultimate fatigue-inducing level of about 40° C. and cause the skin to enter anaerobic metabolism due to decreased perfusion. The device optically detects the increased anaerobic metabolism result of decreased skin perfusion, possibly even with a “normal” reading of SpO₂. The added insight from the skin temperature sensor may also prove to be helpful to guide the user to stay within sustainable levels of exertion. Exercise physiology research may use the device to gain new insights and to more clearly define the normal and abnormal responses of the human body to extreme exertion.

While, in the short term, this blood supply diversion may not be injurious to the person, it does limit the functionality of the relatively oxygen-deprived tissues. Digestion and absorption of nutrients during physical exertion and major illness is progressively limited to immediately absorbable energy sources, such as glucose and fructose, from the gut fluid. Digestion of fats and proteins, on the other hand, requires an array of enzymes, and energy-intensive molecular transport processes, for the absorption of their respective nutrients into the bloodstream; and these processes can only occur efficiently when sufficient oxygen is delivered to the gut tissues. Thus, the device may also serve as a surrogate indicator of perfusion to the gut, possibly lending guidance as to the oxygen availability-dependent aspects of the gut's ability to digest and absorb nutrients.

The advanced alarm algorithm of the device provides the needed security features to guide manual regulation, or to enable automated regulation, of breathing gas oxygen concentration. The professionals attending the patient especially need this function during surgical anesthesia, where the patient's oxygen needs may change either quickly or gradually and, without technology assistance, such changes may not be visually detectable. Another needful, but underserved, area of medical care is during weaning of patients from mechanically assisted ventilation using oxygen enriched breathing gas. The patient's breath tidal volume, the mechanical compliance of the patient's lungs, and the patient's arterial oxygen saturation, or “blood gas” laboratory measurement (SaO₂) and/or pulse oximetry (SpO₂) can usually be adequately monitored by currently available medical equipment. What is lacking, and needed in this life-critical application, is fully reliable, continuous surveillance of the patient's tissue oxygenation status, i.e. ‘is the patient's overall oxygen delivery system adequate to maintain aerobic metabolism even at the skin tissue level?’

An additional clinical care issue is also of importance. Many patients begin to receive medical care intervention when they are already in a compromised physiologic condition. It is of significant clinical importance to know, as soon as possible, the severity of the patient's initial status as this care begins. It is also vital to become aware of when and how rapidly the patient's status improves with intervention, and when the patient's health crisis has been resolved to a normal, stable condition. This entire sequence of information is vital to the initial diagnosis, and to the resulting scaling and continuing management of the level of intervention. Reduction in the level of intervention needs to be made as soon and as quickly as appropriate, while continuing to monitor for relapses, or the onset of new problems.

As mentioned elsewhere herein, when first applied, the initialization sequence of the device sensor will “normalize” at a DC offset of about 10 counts. The LED power levels used to achieve this setting will be recorded as the initial condition. Thereafter, if the patient's skin metabolism becomes more anaerobic, the DC offset will increase, producing an alarm output. On the other hand, as the patient's initially abnormal condition begins to improve, the DC offset will diminish. An ongoing automatic reset will then occur, as needed, to keep the DC offset no less than 10 counts. The changes in LED power level needed to achieve this “recovery” response will be graphically displayed as an index of this improvement. It is anticipated that these LED power level changes, i.e. stepwise increases in the about 805 nm LED power level, will diminish in frequency, and then stabilize at the patient's final fully-treated condition. This index, ultimately, will reveal, by back-projection, the degree of compromise the patient was initially suffering immediately prior to and at the onset of medical care intervention, relative to his/her “normal” aerobic tissue metabolism condition.

The above-described sequence of electronic surveillance will also help to better define the status, recovery milestones and end-points of the need for aggressive, potentially high-risk, interventions. It is now known that intracellular lactic acid levels may remain abnormally elevated for some variable time past the time when the patient's vital signs, i.e. heart rate, blood pressure, SpO₂, and SaO₂ (i.e. arterial blood gas oxygen saturation) have returned to the “normal” range for the patient. It is also known that some recovering patients unexpectedly relapse when support therapies are reduced too soon in response to normalizing “vital signs.” The device presents a new, potentially clinically valuable means of continuously tracking the recovery of patients from tissue hypoxia-related conditions, such as shock, sepsis, heart attack, pneumonia, etc. Those patients, who have not yet fully recovered, at a cellular chemistry level, from the buildup of pyruvate, lactate and NADH due to tissue hypoxia, will potentially benefit from this additional index of their progress. Similarly, patients suffering from obstructive sleep apnea may also accumulate more intracellular products of anaerobic metabolism during sleep than normal, and may take longer to recover normal aerobic metabolism once awakened.

The ECG bio-potential AC signal is capacitively coupled into the input of an internally placed operational amplifier, which amplifies it, delivering the amplified and impedance-reduced signal to one of the sensor solder pads. This amplified ECG signal is then further processed externally of the sensor circuit, to derive a cardiac cycle timing trigger pulse, corresponding to the leading edge of the R-wave of the ECG. This timing trigger pulse is then used to start the control program timers for sampling, sequentially, the following “trough” and “peak” of the PPG. By computing the hemoglobin-oxygen saturation percentage by the now commonly used “ratio-of-ratios” method, heart cycle-related “trough” and “peak” variations in reflected light intensity detection result in a clinically useful determination of SpO₂. The trigger pulse is also used to measure the beat-to-beat time intervals and the associated heart rate by the sensor system.

Some of the more critical and unique needs of newborn premature infants can also be addressed with the device. Just prior to birth, fetal hemoglobin/oxygen saturation of the arterial blood perfusing the fetus's brain and vital organs normally ranges between 55% and 60%. With appropriate initial care of a premature newborn's lungs, especially when the infant is breathing oxygen-enriched breathing gas, the arterial blood oxygen content could become potentially harmful to the retinal blood vessels within a few minutes. Over-oxygenation, also known as oxygen toxicity, of premature infant retinal blood vessels is associated with the development of retro-lental fibroplasia, which causes mechanical distortion and detachment of the infant's retinas, often resulting in permanent visual impairment or even total blindness. On the other hand, too little oxygen delivered to the infant's brain and other vital organs can result in major injuries to these organs and can be fatal. Critical care of premature infants currently has an unmet need for a sensitive, non-invasive, continuous and reliable indicator of the adequacy of oxygen delivery to vital tissues. Using the device's non-invasive, continuous aerobic vs. anaerobic sensing capabilities may provide such a safe and effective guide to help enable greater precision in the care of this very vulnerable population.

Oxygen toxicity is also a recognized problem with older children and adult patients suffering from lung disease. Providing more oxygen than can be consumed is known to result in production and accumulation of free-radical oxygen, which is highly toxic and is known to do significant harm to already compromised lungs and to the blood vessels within the lungs and heart. Unfortunately, the harm from oxygen toxicity is often difficult to distinguish with existing clinical examination and electronic monitoring capabilities from harm caused by excess inflating pressure from a medical ventilator and may be overlooked as a preventable, potentially life-threatening, source of injury.

Thus, the device can continuously register a new, and integrated profile of skin arteriolar SpO₂ and oxygen metabolism, as a sensitive surrogate index of the oxygen supply to vital organs; thus correlating the effects of inhaled oxygen concentration, efficiency of pulmonary gas exchange, cardiac output, and distribution of arterial blood perfusion. The central body placement locations enabled by the sensor format provides improved utility during anesthesia and intensive care and supports effective function even during severe illness conditions that are associated with peripheral vasoconstriction. Detection of persisting intracellular products of anaerobic metabolism may be helpful in guiding the weaning of support during recovery from a variety of severe health challenges, as well as assist with diagnosis and management of chronic problems, such as COPD, heart failure, and obstructive sleep apnea. The device's capacity to effectively bridge over motion artifact signal distortion also enhances utility in high activity applications. Alarm and communications provisions implemented with the device help assure patient safety. The much lower power requirement, compared with currently available pulse oximeter technology, enables use of a smaller battery during ambulatory monitoring.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device comprising:
a first means for emitting a first wavelength wherein the first means for emitting a first wavelength is configurable to emit a first target wavelength during a trough or a peak determined by an ECG R-wave;
a second means for emitting a second wavelength wherein the second means for emitting a second wavelength is configurable to emit a second target wavelength during the trough or the peak determined by the ECG R-wave;
a detection means optically isolated from the first means for emitting the first wavelength and the second means for emitting the second wavelength; and
a processor means configured to receive an input from the detection means.

2.-10. (canceled)

11. A device comprising:
a first LED emitter wherein the first LED emitter is configurable to emit a first target wavelength during a trough or a peak determined by an ECG R-wave;
a second LED emitter wherein the second LED emitter is configurable to emit a second target wavelength during the trough or the peak determined by the ECG R-wave;
a detector optically isolated from the first LED emitter and the second LED emitter; and
a processor configured to receive an input from the detector.

12. The device of claim 11 wherein the first target wavelength is a red wavelength and the second target wavelength is an infrared wavelength.

13. The device of claim 11 wherein the first target center wavelength is 660 nm and the second target center wavelength is 805 nm.

14. The device of claim 11 wherein the device further comprises a data transmitter.

15. The device of claim 11 wherein the device is configurable to detect one or more of reflectance oximetry, and anaerobic metabolism.

16. The device of claim 11 further comprising a housing having a first aperture and a second aperture.

17. The device of claim 16 wherein the first aperture and the second aperture are filled with an optically clear material.

18. The device of claim 11 further comprising an ECG R-wave pre-amplifier circuit.

19. The device of claim 16 further comprising a securer configured to secure the device to an arm or a chest of a user.

20. The device of claim 11 further comprising one or more electrically conductive skin contact adhesive pads.

21. A device comprising:
a housing adapted to engage a chest or an arm of a user wherein the housing has a first aperture and a second aperture;
a first LED emitter wherein the first LED emitter is configurable to emit a first target center wavelength during a trough or a peak determined by an ECG R-wave triggered timing interval through the first aperture;
a second LED emitter wherein the second LED emitter is configurable to emit a second target center wavelength during the trough or the peak determined by the ECG R-wave triggered timing interval through the first aperture;
a detector disposed on an adjacent plate to the LED emitter within the housing wherein the detector is optically isolated in the housing from the first LED emitter and the second LED emitter and adjacent the second aperture; and
a processor configured to receive an input from the detector.

22. The device of claim 21 wherein the first target center wavelength is a red wavelength and the second target center wavelength is an infrared wavelength.

23. The device of claim 21 wherein the first target center wavelength is 660 nm and the second target center wavelength is 805 nm.

24. The device of claim 21 wherein the device further comprises a data transmitter.

25. The device of claim 21 wherein the device is configurable to detect one or more of reflectance oximetry, and anaerobic metabolism.

26. The device of claim 21 wherein the first aperture and the second aperture are filled with an optically clear material.

27. The device of claim 21 further comprising an ECG R-wave pre-amplifier circuit.

28. The device of claim 11 further comprising a securer configured to secure the device to the arm or the chest of the user.

29. A method of detecting a biological parameter comprising:
placing a device in contact with an arm or a chest of a patient wherein the device further comprises, a first LED emitter wherein the first LED emitter is configurable to emit a first target center wavelength during a trough or a peak determined by an ECG R-wave timing trigger and derived timing interval, a second LED emitter wherein the second LED emitter is configurable to emit a second target center wavelength during the trough or the peak determined by the ECG R-wave timing trigger and derived timing interval, a detector optically isolated from the first LED emitter and the second LED emitter, a processor configured to receive an input from the detector,
powering the device with a power supply;
emitting a light in a first wavelength and alternately emitting a light in a second wavelength, wherein the emitted lights are selectively emitted during the trough or peak determined by the ECG-R-wave;
detecting a reflected light; and
analyzing the detected signal produced by the reflected light.

30. The method of claim 29 further comprising the step of determining a reflectance oximetry value for the patient.

31. The method of claim 29 further comprising the step of determining an index of anaerobic metabolism of the sensor-illuminated skin of the patient.

32. The method of claim 29 further comprising the step of transmitting data from the device to a second device.

33. A communication system, comprising:
a detection device having a first LED emitter wherein the first LED emitter is configurable to emit a first target center wavelength during a trough or a peak determined by an ECG R-wave, a second LED emitter wherein the second LED emitter is configurable to emit a second target center wavelength during the trough or the peak determined by the ECG R-wave, a detector optically isolated from the first LED emitter and the second LED emitter, and a detection device processor configured to receive an input from the detector;
a power supply in communication with the detection device to power the detection device;
a server computer system;
a measurement module on the server computer system for permitting a transmission of a measurement from the detection device over a network; and
at least one of an API engine connected to at least one of the detection device to create a message about the measurement and transmit the message over an API integrated network to a recipient having a predetermined recipient user name, an SMS engine connected to at least one of a system for detecting physiological parameters and the detection device to create an SMS message about the measurement and transmit the SMS message over the network to a recipient device having a predetermined measurement recipient telephone number, or an email engine connected to at least one of the detection device to create an email message about the measurement and transmit the email message over the network to a recipient email having a predetermined recipient email address.

34.-39. (canceled)

40. A communication system comprising:
a detection device having a first LED emitter wherein the first LED emitter is configurable to emit a first target center wavelength during a trough or a peak determined by an ECG R-wave, a second LED emitter wherein the second LED emitter is configurable to emit a second target center wavelength during the trough or peak determined by the ECG R-wave, a detector optically isolated from the first LED emitter and the second LED emitter, a detection device processor configured to receive an input from the detector;
a power supply in communication with the detection device to power the detection device;
a server computer system;
a measurement module on the server computer system for permitting a transmission of a measurement from a system for detecting physiological characteristics over a network; and
at least one of an API engine connected to the detection device to create an message about the measurement and transmit the message over an API integrated network to a recipient having a predetermined recipient user name, an SMS engine connected to the detection device to create an SMS message about the measurement and transmit the SMS message over a network to a recipient device having a predetermined measurement recipient telephone number, and an email engine connected to the detection device to create an email message about the measurement and transmit the email message over the network to a recipient email having a predetermined recipient email address.

41.-44. (canceled)
